(19) United States
(12) Patent Application Publication
(10) Pub. No.: US 2004/0014734 A1
(43) Pub. Date: Jan. 22, 2004

(54) Farnesoid X-activated receptor agonists

(76) Inventors: Ching Song, Chicago, IL (US); Shutsung Liao, Chicago, IL (US); Junichi Fukuchi, Chicago, IL (US)

Correspondence Address: FISH & RICHARDSON PC, 225 FRANKLIN ST, BOSTON, MA, 02110, US

(21) Appl. No.: 10/411,889

(22) Filed: Apr. 11, 2003

Related U.S. Application Data
(60) Provisional application No. 60/372,245, filed on Apr. 12, 2002.

(51) Int. Cl. A61K 31/56; C07J 9/00; C07J 41/00

(52) U.S. Cl. 514/169; 552/554; 552/548

(57) ABSTRACT

The invention relates to compounds of formula (I)

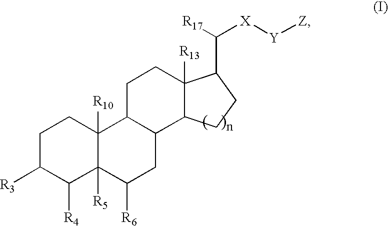

in which n, R₃, R₄, R₅, R₆, R₁₀, R₁₃, R₁₇, X, Y, and Z are defined above. The invention also relates to pharmaceutical compositions each containing an effective amount of one or more compounds of formula (I) and a pharmaceutically acceptable carrier.

### CROSS REFERENCE TO RELATED APPLICATIONS

This application claims benefit of U.S. Provisional Application Serial No. 60/372,245, filed Apr. 12, 2002.

### FUNDING

Work described herein was supported by a grant from the National Institute of Health (CA-58073). The U.S. government has certain rights in the invention.

### BACKGROUND

Bile acids (BA) are water-soluble end products of cholesterol metabolism and play an important role in human physiology. For instance, they participate in body cholesterol disposal as well as generation of bile flow and biliary lipid secretion through transcriptional activation of several genes involved in the conversion of cholesterol into BA. See, e.g., Hofmann, Arch. Intern. Med., 1999, 159, 2647-2658.

Farnesoid X-activated receptor (FXR) is a BA activated nuclear receptor that regulates expression of BA responsive target genes in the intestine and liver. See, e.g., Makishima et al., Science, 1999, 284, 362-365; and Parks et al., Science, 1999, 1365-1368. Knockout mice exhibited elevated levels of cholesterol and triglyceride (including triglyceride-rich lipoproteins), and decreased BA excretion. See, e.g., Edwards, et al., J. Lipid Res., 2002, 43, 2-12. Thus, FXR agonists would be promising drugs for treating disorders related to elevated cholesterol or triglyceride levels.

### SUMMARY

One aspect of this invention relates to compounds of formula (I):

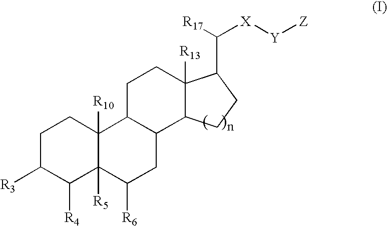

In the above formula, n is 1 or 2; each of R₃, R₄, R₅, and R₆, independently, is hydrogen; each of R₁₀, R₁₃, and R₁₇, independently, is hydrogen or C₁₋₄ alkyl; X is C₁₋₈ alkylene, C₂₋₈ alkenylene, or C₂₋₈ alkynylene; Y is —CO—, —CS—, or —CNH—; and Z is C₁₋₈ alkyl or NRₐR_{b}, wherein each of Rₐ and R_{b}, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl. Examples of these compounds include those in which n is 1; those in which each of R₁₀, R₁₃, and R₁₇, independently, is methyl; those in which Y is —CO—; those in which X is C₁₋₄ alkylene (e.g., ethylene); and those in which Z is (methyl)(carboxymethyl)-amino, (methyl)(methoxy)amino, (carboxymethyl)amino, (methylamino)(methyl)amino, (methylamino)(methyl)amino, (formylamino)amino, (2,2,2-trifluoroethyl)amino, dimethylamino, ethyoxycarbomethyl, or (2-chloroethyl)amino.

Another aspect of this invention relates to compounds also of formula (I), in which n, R₁₀, R₁₃, R₁₇, and X are defined as above; each of R₃, R₄, R₅, and R₆, independently, is hydrogen; or R₃ and R₄ together, or R₅ and R₆ together, are eliminated so that the carbon atoms to which they are attached are connected via a double bond; Y is —SO—, —SO₂—, —PO—, or —PO₂—; Z is hydroxyl, C₁₋₈ alkyl, C₁₋₈ alkoxy, or NRₐR_{b}, wherein each of Rₐ and R_{b}, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl. Examples of these compounds include those in which n is 1; those in which each of R₃, R₄, R₅, and R₆, independently, is hydrogen; and those in which each of R₁₀, R₁₃, and R₁₇, independently, is methyl; those in which X is C₁₋₈ alkylene (e.g., ethylene); those in which Y is —SO₂—; and those in which Z is hydroxy.

Still another aspect of this invention relates to a compound also of formula (I) in which n, R₁₀, R₁₃, R₁₇, and X are also defined as above; R₃ and R₄ together, or R₅ and R₆ together, are eliminated so that the carbon atoms to which they are attached are connected via a double bond; Y is —CO—, —CS—, —CNH—, —SO—, —SO₂—, —PO—, or —PO₂—; Z is hydroxyl, C₁₋₈ alkyl, C₁₋₈ alkoxy, or NRₐR_{b}, wherein each of Rₐ and R_{b}, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl. Examples of these compounds include those in which n is 1; those in which each of R₁₀, R_{13,} and R_{17,} independently, is methyl; those in which X is C₁₋₈ alkylene (e.g., ethylene); those in which Y is —CO—; and those in which Z is hydroxy.

The terms “alkyl,” the prefix “alk” (e.g., as in “alkoxy”), and the suffix “-alkyl” (e.g., as in “Sulfonylalkyl”) mentioned above all refer to linear or branched alkyl moieties. The terms “alkylene,” “alkenylene,” and “alkylnylene” respectively refer to divalent radicals of alkyl (e.g., —CH₂—), alkenyl (e.g., —CH₂—CH═CH—CH₂—), and alkynyl (e.g., —CH₂—C-≡C—CH₂—).

Specific examples of the compounds of this invention include N-methyl-N-carboxymethyl-5β-cholanoic acid-24-amide, N-methyl-N-methoxy-5β-cholanoic acid-24-amide, N-carboxymiiethyl-5β-cholanoic acid-24-amide, N-sulfonylethyl-5β-cholaioic acid-24-amide, N-metliyl-N-nmethylamiio-5β-cholanoic acid-24-amide, N,N-dimethyl-5β-cholanoic acid-24-amide, N-methyl-5β-cholanoic acid-24-amide, N-(2-chloroethyl)-5β-cholanoic acid-24-amide, 5β-cholanoic acid methyl ester, and Δ^{3,5}-5β-cholanoic acid.

The compounds described above also include their pharmaceutically acceptable salts and prodrugs. The compounds above further include an ester, an amide, an enantiomer, an isomer, a tautomer, a polymorph, or a derivative thereof, if applicable. Such salts, for example, can be formed by interaction between a positively charged substituent on a compound of this invention (e.g., amino) and an anion. Suitable anions include, but are not limited to, chloride, bromide, iodide, sulfate, nitrate, phosphate, citrate, methanesulfonate, trifluoroacetate, and acetate. Likewise, a negatively charged substituent in a compound of this invention (e.g., carboxylate) can form a salt with a cation. Suitable cations include, but are not limited to, sodium ion, potassium ion, magnesium ion, calcium ion, and an ammonium cation such as teteramethylammonium ion. Examples of prodrugs include esters and other pharmaceutically acceptable derivatives, which, upon administration to a subject, are capable of providing cholanoic acid compounds described above.

Yet still another aspect of this invention relates to a pharmaceutical composition which includes a pharmaceutically acceptable carrier and an effective amount of a compound of formula (I) shown above. In these compounds, n is 1 or 2; each of R₃, R₄, R₅, and R₆, independently, is hydrogen; or R₃ and R₄ together, or R₅ and R₆ together, are eliminated so that the carbon atoms to which they are attached are connected via a double bond; each of R₁₀, R₁₃, and R₁₇, independently, is hydrogen or C₁₋₄ alkyl; X is C₁₋₈ alkylene, C₂₋₈ alkenylene, or C₂₋₈ alkynylene; Y is —CO—, —CS—, —CNH—, —SO—, —SO₂—, —PO—, or —PO₂—; and Z is hydroxyl, C₁₋₈ alkyl, C₁₋₈ alkoxy, or NR₁R₂, wherein each of R₁ and R₂, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl.

The compounds that are contained in the pharmaceutical compositions of this invention include those in which n is 1; those in which each of R₃, R₄, R₅, and R₆, independently, is hydrogen; those in which R₃ and R₄ together, and R₅ and R₆ together, are eliminated so that the carbon atoms to which they are attached are connected via a double bond; those in which each of R₁₀, R₁₃, and R₁₇, independently, is methyl; those in which Y is —CO— or —SO₂—; those in which X is C₁₋₈ alkylene (e.g., ethylene); those in which Z is (methyl)(carboxymethyl)amino, (methyl)(methoxy)amino, (carboxymethyl)amino, (methylamino)(methyl)amino, (methylamino)(methyl)amino, (formylamino)amino, (2,2,2-trifluoroethyl)amino, dimethylamino, ethyoxycarbomethyl, (2-chloroethyl)amino, or hydroxy.

The compounds of this invention are agonists of FXR and therefore can be used to treat FXR-mediated disorders such as hypercholesteromenia and hypertriglycerimenia. Thus, within the scope of this invention is a method of treating an FXR-mediated disorder (e.g., a disorder related to a high cholesterol or triglyceride levels in blood). The method includes administering to a subject in need thereof an effective amount of a compound of this invention. Also within the scope of this invention is a method of lowering the cholesterol or triglyceride levels in the blood. This method includes contacting a compound of this invention with the FXR in liver cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Details of several compounds of this invention are set forth in the accompanying description below. Other features, objects, and advantages of this invention will be apparent from the description and from the claims.

### DETAILED DESCRIPTION

Compounds that can be used to practice the methods, kits, combinations, and compositions of this invention can be synthesized by methods well known in the art with a suitable steroidal derivative as a starting material. More specifically, such a steroidal derivative possesses a substituent at C-17 of the steroidal ring system that can be modified to contain a moiety defined by X, Y, and Z [shown in formula (I)]. Examples of such steroidal derivatives include cholic acid, cholanoic acid, 3α, 6α-dihydroxy-5β-cholanoic acid 24-methyl ester, ursodeoxycholic acid, and hyochlolic acid. They are either commercially available or can be synthesized by methods described in the literature, e.g., Roda et al., F. Lipid Res., 1994, 35: 2268-2279; and Roda et al., Dig. Dis. Sci., 1987, 34: 24S-35S.

A compound of this invention that has an amide-containing substitutent at C-17 (i.e., Y is —CO—, and Z is —NRₐR_{b}) can be prepared by reacting a steroidal derivative having a carbonyl- or carboxyl-containing substituting at C-17 (e.g., cholanoic acid) with an amino-containing compound (such as dimethylamine, aniline, glycine, and phenylalanine). Similarly, a compound of this invention that has an ester-containing substitutent at C-17 (i.e., Y is —CO—, and Z is alkoxy) can be prepared by reacting a steroidal derivative having a carbonyl or carboxy-containing substituent at C-17 with a hydroxyl-containing compound (such as ethanol and isopropanol). The amide- or ester-forming reaction can take place in any suitable solvent. If the reaction takes place in an aqueous solution, isolation of the steroid product for in vitro or in vivo screening assays may not be necessary.

A compound of this invention that has a carbonyl-containing substitutent at C-17 (i.e., Y is —CO—) can be converted, e.g., to a thiocarbonyl-containing compound of this invention (i.e., Y is —CS—) by reacting it with sulfur hydride, or to an imino-containing compound of this invention (i.e., Y is —CNH—) by reacting it with a hydrazinie. See Janssen et al. (Ed.), Organosulfur Chemistry; Wiley: New York, 1967, 219-240; and Patai et al. (Ed.), The Chemistry of the Carbon-Nitrogen Double Bond; Wiley: New York, 1970, 64-83 and 465-504, respectively.

A compound of this invention that has a sulfonyl- or phosphoryl-containing substituents at C-17 can be prepared by reacting a steroidal derivative having a halogen-containing substituent at C-17 with a phosphate compound or sulfate compound (e.g., soldium sulfate).

Due to the simplicity of the reaction, it can be easily automated. Isolation and quantification of the product can be done by thin-layer chromatography, high pressure liquid chromatography, gas chromatography, capillary electrophoresis, or other analytical and preparative procedures.

The term “treat” or “treatment” as used herein refers to any treatment of a disorder or disease associated with a disease or disorder related to high blood serum concentration of cholesterol or triglycerides in a subject, and includes, but is not limited to, preventing the disorder or disease from occurring in a subject which may be predisposed to the disorder or disease, but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

The term “prevent” or “prevention,” in relation to a disease or disorder related to high blood serum concentration of cholesterol or triglyceride in a subject, means no disease or disorder development if none had occurred, or no further disorder or disease development if there had already been development of the disorder or disease.

The phrase “high blood serum concentration of cholesterol” or “high blood serum cholesterol concentration” as used herein refers to cholesterol blood serum levels in a subject that is generally above that which has generally been determined healthy or normal, and is, or can lead to the development of a disease or disorder associated with high serum concentrations of cholesterol. The phrase “high blood serum concentration of triglycerides” or “high blood serum triglyceride concentration” as used herein refers to triglyceride blood serum levels in a subject that is generally above that which has generally been determined healthy or normal, and is, or can lead to the development of a disease or disorder associated with high serum concentrations of triglyceride. The healthy or normal level will vary from species to species and even subject to subject, or be age specific, for example, however, a person of ordinary skill in the art will be able to determine a healthy or normal level for each subject. Healthy or normal levels of cholesterol or triglyceride can be calculated by referencing many scientific and medical publications. Generally, cholesterol is measured in a subject as total plasma cholesterol, LDL cholesterol and HDL cholesterol and triglyceride levels are measured as total plasma triglycerides. Illustratively, in an adult human, high blood serum cholesterol concentration is generally considered to be above about 5.2 mmol/L (200 mg/dL) for total plasma cholesterol; and/or above about 3.36 mmol/L (130 mg/dL) for LDL cholesterol; high serum triglyceride concentration is generally considered to be above about 1.69 mmol/L (150 mg/dL). In another embodiment, in an adult human, high blood serum cholesterol concentration is generally considered to be above about 5.2 to about 6.18 mmol/L (200-239 mg/dL) for total plasma cholesterol; and/or above about 3.36 to about 4.11 mmol/L (130-159 mg/dL) for LDL cholesterol; high serum triglyceride concentration is generally considered to be above about 1.69 to about 2.24 mmol/L (150-199 mg/dL). In yet another embodiment, in an adult human, high blood serum cholesterol concentration is generally considered to be above about 6.21 mmol/L (240 mg/dL) for total plasma cholesterol; and/or above about 4.14 mmol/L (160 mg/dL) for LDL cholesterol level; high serum triglyceride concentration is generally considered to be above about 2.25 mmol/L (200 mg/dL).

An effective amount of a compound thus prepared can be formulated with a pharmaceutically acceptable carrier to form a pharmaceutical composition before being administered for treatment of a disorder related to an elevated level of cholesterol or triglyceride. “An effective amount” refers to the amount of the compound which is required to confer therapeutic effect on the treated subject. The interrelationship of dosages for animals and humans (based on milligrams per square meter of body surface) is described by Freireich et al., Cancer Chemother. Rep. 1966, 50, 219. Body surface area may be approximately determined from height and weight of the patient. See, e.g., Scientific Tables, Geigy Pharmaceuticals, Ardley, New York, 1970, 537. Effective doses will also vary, as recognized by those skilled in the art, depending on the route of administration, the excipient usage, and the optional co-usage with other therapeutic treatments. Examples of pharmaceutically acceptable carriers include colloidal silicon dioxide, magnesium stearate, cellulose, sodium lauryl sulfate, and D&C Yellow # 10.

Toxicity and therapeutic efficacy of the active ingredients can be determined by standard pharmacutical procedures, e.g., for determining LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Included in the methods, kits, combinations and pharmaceutical compositions of the present invention are the isomeric forms and tautomers of the described compounds and the pharmaceutically-acceptable salts thereof Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

The term “prodrug” refers to a drug or compound in which the pharmacological action (active curative agent) results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone- beta -D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard [Int. J. Pharmaceutics, 37, 87 (1987)] describe the evaluation of N-acylsulfonamides as potential prodrug derivatives. J. Larsen et al., [Int. J. Pharmaceutics, 47, 103 (1988)] describe the evaluation of N-methylsulfonamides as potential prodrug derivatives. Prodrugs are also described in, for example, Sinkula et al., J. Pharm. Sci., 64:181-210 (1975).

The term “derivative” refers to a compound that is produced from another compound of similar structure by the replacement of substitution of one atom, molecule or group by another. For example, a hydrogen atom of a compound may be substituted by alkyl, acyl, amino, etc., to produce a derivative of that compound.

“Plasma concentration” refers to the concentration of a substance in blood plasma or blood serum.

“Drug absorption” or “absorption” refers to the process of movement from the site of administration of a drug toward the systemic circulation, for example, into the bloodstream of a subject.

“Bioavailability” refers to the extent to which an active moiety (drug or metabolite) is absorbed into the general circulation and becomes available at the site of drug action in the body.

“Metabolism” refers to the process of chemical alteration of drugs in the body.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Half-life” refers to the time required for the plasma drug concentration or the amount in the body to decrease by 50% from its maximum concentration.

The use of the term “about” in the present disclosure means “approximately,” and illustratively, the use of the term “about” indicates that dosages outside the cited ranges may also be effective and safe, and such dosages are also encompassed by the scope of the present claims.

The term “measurable serum concentration” means the serum concentration (typically measured in mg, μg, or ng of therapeutic agent per ml, dl, or l of blood serum) of a therapeutic agent absorbed into the bloodstream after administration.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal (Group Ia) salts, alkaline earth metal (Group IIa) salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimetlylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like. The compositions of the present invention are usually administered in the form of pharmaceutical compositions. These compositions can be administered by any appropriate route including, but not limited to, oral, rectal, transdermal, parenteral (for example, subcutaneous, intramuscular, intravenous, intramedullary, intraperitoneal, and intradermal injections, or infusion techniques administration), intranasal (for example, nasogastric tube), transmucosal, implantation, inhalation spray, vaginal, topical, and buccal (for example, sublingual). Such preparations may routinely contain buffering agents, preservatives, penetration enhancers, compatible carriers and other therapeutic ingredients. Examples of parenteral dosage forms include an active compound dissolved in a phosphate buffer solution, or admixed with any other pharmaceutically acceptable carrier. Solubilizing agents, such as cyclodextrins, or other solubilizing agents well known to those familiar with the art, can also be included in the pharmaceutical composition.

The present invention also includes methods employing a pharmaceutical composition that contains the composition of the present invention associated with pharmaceutically acceptable carriers or excipients. As used herein, the terms “pharmaceutically acceptable carrier” or “pharmaceutically acceptable excipients” includes any and all solvents, dispersion media, coatings, antibacterial and antifungal agents, isotonic and absorption delaying agents, and the like. The use of such media and agents for ingestible substances is well known in the art. Except insofar as any conventional media or agent is incompatible with the compositions, its use is contemplated. Supplementary active ingredients can also be incorporated into the compositions.

In making the compositions of the present invention, the compositions(s) can be mixed with a pharmaceutically acceptable excipient, diluted by the excipient or enclosed within such a carrier, which can be in the form of a capsule, sachet, paper or other container. The carrier materials that can be employed in making the composition of the present invention are any of those commonly used excipients in pharmaceutics and should be selected on the basis of compatibility with the active drug and the release profile properties of the desired dosage forms. Illustratively, a pharmaceutical excipient except active drugs are chosen below as examples:

(a) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

(b) Disintegration agents such as starches, pregelatinized corn starch, pregelatinized starch, celluloses, cross-linked carboxymethylcellulose, sodium starch glycolate, crospovidone, cross-linked polyvinylpyrrolidone, croscarmellose sodium, a calcium, a sodium alginate complex, clays, alginates, gums, or sodium starch glycolate, and any disintegration agents used in tablet preparations.

(c) Filling agents Such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

(d) Surfactants Such as sodium lauryl sulfate, sorbitan monooleate, polyoxyethylene sorbitan monooleate, polysorbates, polaxomers, bile salts, glyceryl monostearate, Pluronic™ line (BASF), and the like.

(e) Solubilizer such as citric acid, succinic acid, fumaric acid, malic acid, tartaric acid, maleic acid, glutaric acid sodium bicarbonate and sodium carbonate and the like.

(f) Stabilizers such as any antioxidation agents, buffers, or acids, and the like, can also be utilized.

(g) Lubricants such as magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

(h) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

(i) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

(j) Anti-adherenits or glidants such as talc, corn starch, DL-leucine, sodium lauryl sulfate, and magnesium, calcium, or sodium stearates, and the like.

(k) Pharmaceutically compatible carrier comprises acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, caltageenan, monoglyceride, diglyceride, or pregelatinized starch, and the like.

Additionally, drug formulations are discussed in, for example, Hoover, John E., Remington's The Science and Practice of Pharmacy (2000). Another discussion of drug formulations can be found in Liberman, H. A. and Lachman, L., Eds., Pharmaceutical Dosage Forms, Marcel Decker, New York, N.Y., 1980.

Besides being useful for human treatment, the present invention is also useful for other subjects including veterinary animals, reptiles, birds, exotic animals and farm animals, including mammals, rodents, and the like. Mammal includes a primate, for example, a monkey, or a lemur, a horse, a dog, a pig, or a cat. A rodent includes a rat, a mouse, a squirrel, or a guinea pig.

An in vitro assay can be conducted to preliminarily screen a compound of this invention for its efficacy in agonizinig FXR and thus in treating an FXR-mediated disease. For instance, human embryonic cells are transfected with a luciferase reporter gene (which includes a human c-fos minimal promoter) and FXR. After incubating the transfected cells with a compound to be tested, the activity of luciferase is measured to determine the transactivation extent of the reporter gene. See, e.g., Janowski et al., Nature, 1996, 383, 728-731; Hong, 1996, 2987; and Chiang, 1994.

Compounds that show efficacy in the preliminary assay can be further evaluated in an animal study by a method also well known in the art. For example, a compound can be orally administered to mice fed with a cholesterol-containing diet. The efficacy of the compound can be determined by comparing cholesterol levels in various tissues of the treated mice with those in non-treated mice.

For treatment of a FXR-mediated disorder, disease, or disease symptom, compositions of the invention can be used to provide a dose of a compound of the present invention of about 5 ng to about 1000 mg, or about 100 ng to about 600 mg, or about 1 mg to about 500 mg, or about 20 mg to about 400 mg. A dose can be administered in one to about four doses per day, or in as many doses per day to elicit a therapeutic effect. Illustratively, a dosage unit of a composition of the present invention can typically contain, for example, about 5 ng, 50 ng 100 ng, 500 ng, 1 mg, 10 mg, 20 mg, 40 mg, 80 mg, 100 mg, 125 mg, 150 mg, 200 mg, 250 mg, 300 mg, 350 mg, 400 mg, 450 mg, 500 mg, 550 mg, 600 mg, 700 mg, 800 mg, 900 mg, or 1000 mg of a compound of the present invention. The dosage form can be selected to accommodate the desired frequency of administration used to achieve the specified dosage. The amount of the unit dosage form of the composition that is administered and the dosage regimen for treating the condition or disorder depends on a variety of factors, including, the age, weight, sex and medical condition, of the subject, the severity of the condition or disorder, the route and frequency Of administration, and this can vary widely, as is well known.

In one embodiment of the present invention, the composition is administered to a subject in an effective amount, that is, the composition is administered in an amount that achieves a therapeutically-effective dose of a compound of the present invention in the blood serum of a subject for a period of time to elicit a desired therapeutic effect. Illustratively, in a fasting adult human (fasting for generally at least 10 hours) the composition is administered to achieve a therapeutically-effective dose of a compound of the present invention in the blood serum of a subject from about 5 minutes after administration of the composition. In another embodiment of the present invention, a therapeutically-effective dose of the compound of the present invention is achieved in the blood serum of a subject at about 10 minutes from the time of administration of the composition to the subject. In another embodiment of the present invention, a therapeutically-effective dose of the compound of the present invention is achieved in the blood serum of a subject at about 20 minutes from the time of administration of the composition to the subject. In yet another embodiment of the present invention, a therapeutically-effective dose of the compound of the present invention is achieved in the blood serum of a subject at about 30 minutes from the time of administration of the composition to the subject. In still another embodiment of the present invention, a therapeutically-effective dose of the compound of the present invention is achieved in the blood serum of a subject at about 40 minutes from the time of administration of the composition to the subject. In one embodiment of the present invention, a therapeutically-effective dose of the compound of the present invention is achieved in the blood serum of a subject at about 20 minutes to about 12 hours from the time of administration of the composition to the subject. In another embodiment of the present invention, a therapeutically-effective dose of the compound of the present invention is achieved in the blood serum of a subject at about 20 minutes to about 6 hours from the time of administration of the composition to the subject. In yet another embodiment of the present invention, a therapeutically-effective dose of the compound of the present invention is achieved in the blood serum of a subject at about 20 minutes to about 2 hours from the time of administration of the composition to the subject. In still another embodiment of the present invention, a therapeutically-effective dose of the compound of the present invention is achieved in the blood serum of a subject at about 40 minutes to about 2 hours from the time of administration of the composition to the subject. And in yet another embodiment of the present invention, a therapeutically-effective dose of the compound of the present invention is achieved in the blood serum of a subject at about 40 minutes to about 1 hour from the time of administration of the composition to the subject.

In one embodiment of the present invention, a composition of the present invention is administered at a dose suitable to provide a blood serum concentration with a half maximum dose of a compound of the present invention. Illustratively, a blood serum concentration of about 0.01 to about 1000 nM, or about 0.1 to about 750 nM, or about 1 to about 500 nM, or about 20 to about 1000 nM, or about 100 to about 500 nM, or about 200 to about 400 nM is achieved in a subject after administration of a composition of the present invention. Contemplated compositions of the present invention provide a therapeutic effect as compound of the present invention medications over an interval of about 5 minutes to about 24 hours after administration, enabling once-a-day or twice-a-day administration if desired. In one embodiment of the present invention, the composition is administered at a dose suitable to provide an average blood serum concentration with a half maximum dose of a compound of the present invention of at least about 1 μg/ml; or at least about 5 μg/ml, or at least about 10 μg/ml, or at least about 50 μg/ml, or at least about 100 μg/ml, or at least about 500 μg/ml, at least about 1000 μg/ml in a subject about 10, 20, 30, or 40 minutes after administration of the composition to the subject.

The amount of therapeutic agent necessary to elicit a therapeutic effect can be experimentally determined based on, for example, the absorption rate of the agent into the blood serum, the bioavailability of the agent, and the potency for modulating a farnesoid X receptor. It is understood, however, that specific dose levels of the therapeutic agents of the present invention for any particular subject depends upon a variety of factors including the activity of the specific compound employed, the age, body weight, general health, sex, and diet of the subject (including, for example, whether the subject is in a fasting or fed state), the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route administered, the condition of the particular subject, etc. Generally speaking, one will desire to administer an amount of the compound that is effective to achieve a serum level commensurate with the concentrations found to be effective in vitro for a period of time effective to elicit a therapeutic effect. Thus, where a compound is found to demonstrate in vitro activity at, for example, a half-maximum effective dose of 200 nM, one will desire to administer an amount of the drug that is effective to provide about a half-maximum effective dose of 200 nM concentration in vivo for a period of time that elicits a desired therapeutic effect, for example, agonizing a farnesoid X receptor, and thus treating an FXR-mediated disorder, disease, or disease symptom related to high cholesterol or triglyceride concentration, e.g., treating arteriosclerosis, treating a senile cognitive impairment, treating dementia, treating Alzheimer's, and other indicators as are selected as appropriate measures by those skilled in the art. Determination of these parameters is well within the skill of the art. These considerations are well known in the art and are described in standard textbooks.

In order to measure and detenrine the effective amount of a compound of the present invention to be delivered to a subject, serum compound of the present invention concentrations can be measured using standard assay techniques.

Contemplated compositions of the present invention provide a therapeutic effect over an interval of about 30 minutes to about 24 hours after administration to a subject. In one embodiment compositions provide such therapeutic effect in about 30 minutes. In another embodiment compositions provide therapeutic effect over about 24 hours, enabling once-a-day administration.

In another aspect, the present invention is directed to therapeutic methods of treating a condition or disorder where treatment with a farnesoid X receptor agonist is indicated, the method comprises the oral administration of one or more compositions of the present invention to a subject in need thereof. In one embodiment, the condition or disorder is a vascular disorder or a neurodegenerative disorder.

The present methods, kits, and compositions can also be used in combination (“combination therapy”) with another pharmaceutical agent that is indicated for treating or preventing a vascular disorder or a neurodegenerative disorder, such as, for example, a statin (e.g., lovastatin) an angiotensin converting enzyme inhibitor, an angiotensin II receptor antagonist, an antianthythmic, an anticholersteremic, a diuretic, a dopamine receptor agonist, a dopamine receptor antagonist, or a vasodilator, which are commonly administered to treat, prevent, or minimize the symptoms and complications related to this disorder. These drugs have certain disadvantages associated with their use. Some of these drugs are not completely effective in the treatment of the aforementioned conditions and/or produce adverse side effects, such as mental confusion, constipation, diarrhea, etc. However, when used in conjunction with the present invention, that is, in combination therapy, many if not all of these unwanted side effects can be reduced or eliminated. The reduced side effect profile of these drugs is generally attributed to, for example, the reduce dosage necessary to achieve a therapeutic effect with the administered combination.

The phrase “combination therapy” embraces the administration of a composition of the present invention in conjunction with another pharmaceutical agent that is indicated for treating or preventing a vascular disorder or a neurodegenerative disorder in a subject, as part of a specific treatment regimen intended to provide a beneficial effect from the co-action of these therapeutic agents for the treatment of a vascular disorder or a neurodegenerative disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. The composition of the present invention can be administered orally or nasogastric, while the other therapeutic agent of the combination can be administered by any appropriate route for that particular agent, including, but not limited to, an oral route, a percutaneous route, an intravenous route, an intramuscular route, or by direct absorption through mucous membrane tissues. For example, the composition of the present invention is administered orally or nasogastric and the therapeutic agent of the combination may be administered orally, or percutaneously. The sequence in which the therapeutic agents are administered is not narrowly critical. “Combination therapy” also can embrace the administration of the therapeutic agents as described above in further combination with other biologically active ingredients, such as, but not limited to, an analgesic, for example, and with non-drug therapies, such as, but not limited to, surgery.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally (for example, sublingual), or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

For oral administration, the pharmaceutical composition can contain a desired amount of a farnesoid X receptor agonist and be in the form of, for example, a tablet, a hard or soft capsule, a lozenge, a cachet, a dispensable powder, granules, a suspension, an elixir, a liquid, or any other form reasonably adapted for oral administration. Illustratively, such a pharmaceutical composition can be made in the form of a discrete dosage unit containing a predetermined amount of the farnesoid X receptor agonist such as a tablet or a capsule. Such oral dosage forms can further comprise, for example, buffering agents. Tablets, pills and the like additionally can be prepared with enteric coatings.

Pharmaceutical compositions suitable for buccal (sublingual) administration include, for example, lozenges comprising a farnesoid X receptor agonist in a flavored base, such as sucrose, and acacia or tragacanth, and pastilles comprising a farnesoid X receptor agonist in an inert base such as gelatin and glycerin or sucrose and acacia.

Liquid dosage forms for oral administration can include pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

Examples of suitable liquid dosage forms include, but are not limited, aqueous solutions comprising a farnesoid X receptor agonist and beta-cyclodextrin or a water soluble derivative of beta-cyclodextrin such as sulfobutyl ether beta-cyclodextrin; heptakis-2,6-di-O-methyl-beta-cyclodextrin; hydroxypropyl-beta-cyclodextrin; and dimethyl-beta-cyclodextrin.

The pharmaceutical compositions of the present invention can also be administered by injection (intravenous, intramuscular, subcutaneous). Such injectable compositions can employ, for example, saline, dextrose, or water as a suitable carrier material. The pH value of the composition can be adjusted, if necessary, with suitable acid, base, or buffer. Suitable bulking, dispersing, wetting or suspending agents, including mannitol and polyethylene glycol (such as PEG 400), can also be included in the composition. A suitable parenteral composition can also include a farnesoid X receptor agonist in injection vials. Aqueous solutions can be added to dissolve the composition prior to injection.

The pharmaceutical compositions can be administered in the form of a suppository or the like. Such rectal formulations preferably contain a farnesoid X receptor agonist in a total amount of, for example, about 0.075 to about 75% w/w, or about 0.2 to about 40% w/w, or about 0.4 to about 15% w/w. Carrier materials such as cocoa butter, theobroma oil, and other oil and polyethylene glycol suppository bases can be used in such compositions. Other carrier materials such as coatings (for example, hydroxypropyl methylcellulose film coating) and disintegrants (for example, croscarmellose sodium and cross-linked povidone) can also be employed if desired.

These phannaceutical compositions can be prepared by any suitable method of pharmacy which includes the step of bringing into association a famesoid X receptor agonist of the present invention and a carrier material or carriers materials. In general, the compositions are uniformly and intimately admixing the active compound with a liquid or finely divided solid carrier, or both, and then, if necessary, shaping the product. For example, a tablet can be prepared by compressing or molding a powder or granules of the compound, optionally with one or more accessory ingredients. Compressed tablets can be prepared by compressing, in a suitable machine, the compound in a free-flowing form, such as a powder or granules optionally mixed with a binding agent, lubricant, inert diluent and/or surface active/dispersing agent(s). Molded tablets can be made by molding, in a suitable machine, the powdered compound moistened with an inert liquid diluent.

Tablets of the present invention can also be coated with a conventional coating material such as Opadry™ White YS-1-18027A (or another color) and the weight fraction of the coating can be about 3% of the total weight of the coated tablet. The compositions of the present invention can be formulated so as to provide quick, sustained or delayed release of the compositions after administration to the patient by employing procedures known in the art.

When the excipient serves as a diluent, it can be a solid, semi-solid or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), soft and hard gelatin capsules and sterile packaged powders.

Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents and pharmaceutically compatible carriers. Such tablets may also comprise film coatings, which dissolve upon oral ingestion or upon contact with diluent.

In one embodiment of the present invention, the manufacturing processes may employ one or a combination of methods including: (1) dry mixing, (2) direct compression, (3) milling, (4) dry or non-aqueous granulation, (5) wet granulation, or (6) fusion. Lachman et al., The Theory and Practice of Industrial Pharmacy (1986).

In another embodiment of the present invention, solid compositions, such as tablets, are prepared by mixing a therapeutic agent of the present invention with a pharmaceutical excipient to form a solid preformulation composition containing a homogeneous mixture of the therapeutic agent and the excipient. When referring to these preformulation compositions(s) as homogeneous, it is meant that the therapeutic agent is dispersed evenly throughout the composition so that the composition may be readily subdivided into equally effective unit dosage forms, such as tablets, pills and capsules. This solid preformulation is then subdivided into unit dosage forms of the type described herein.

Compressed tablets are solid dosage forms prepared by compacting a formulation containing an active ingredient and excipients selected to aid the processing and improve the properties of the product. The term “compressed tablet” generally refers to a plain, uncoated tablet for oral ingestion, prepared by a single compression or by pre-compaction tapping followed by a final compression.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. A variety of materials can be used for such enteric layers or coatings, including a number of polymeric acids and mixtures of polymeric acids with such materials as shellac, cetyl alcohol and cellulose acetate.

The term “suspension tablets” as used herein refers to compressed tablets which rapidly disintegrate after they are placed in water, and are readily dispersible to form a suspension containing a precise dose of the compositions(s). Croscarmellose sodium is a known disintegrant for tablet formulations, and is available from FMC Corporation, Philadelphia, Pa. under the trademark Ac-Di-Sol®. It is frequently blended in compressed tableting formulations either alone or in combination with microcrystalline cellulose to achieve rapid disintegration of the tablet.

Microcrystalline cellulose, alone or co-processed with other ingredients, is also a common additive for compressed tablets and is well known for its ability to improve compressibility of difficult to compress tablet materials. It is well known in the art that commercially available products are available and can be used with the present invention. One example is available under the Avicel® trademark. Two different Avicel® products are utilized, Avicel® PH which is microcrystalline cellulose, and Avicel® AC-815, a co processed spray dried residue of microcrystalline cellulose and a calcium-sodium alginate complex in which the calcium to sodium ratio is in the range of about 0.40:1 to about 2.5:1. While AC-815 is comprised of 85% microcrystalline cellulose (MCC) and 15% of a calcium-sodium alginate complex, for purposes of the present invention this ratio may be varied from about 75% MCC to 25% alginate up to about 95% MCC to 5% alginate. Depending on the particular formulation and active ingredient, these two components may be present in approximately equal amounts or In unequal amounts, and either may comprise from about 10% to about 50% by weight of the tablet.

Dry oral formulations can contain such excipients as binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials) and lubricating agents (for example, stearates and talc).

Since the tablet may be used to form rapidly disintegrating chewable tablets, lozenges, troches or swallowable tablets; the intermediate formulations, as well as the process for preparing them, provide additional aspects of the present invention.

Effervescent tablets and powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid.

When the salts are added to water, the acids and the base react to liberate carbon dioxide gas, thereby causing “effervescence.”

The method of preparation of the effervescent granules of the present invention employs three basic processes: wet granulation, dry granulation and fusion. The fusion method is used for the preparation of most commercial effervescent powders. It should be noted that, although these methods are intended for the preparation of granules, the formulations of effervescent salts of the present invention could also be prepared as tablets, according to well-known prior art technology for tablet preparation.

Wet granulation is the oldest method of granule preparation. The individual steps in the wet granulation process of tablet preparation include milling and sieving of the ingredients, dry powder mixing, wet massing, granulation and final grinding.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

In another aspect, the present invention is directed to therapeutic methods of treating a condition or disorder where treatment with a farnesoid X receptor agonist is indicated, the method comprises the oral administration of one or more compositions of the present invention to a subject in need thereof. In one embodiment, the condition or disorder is a vascular disorder or a neurodegenerative disorder.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The pharmaceutically acceptable carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such a lecithin, by the maintenance of the required particle size in the case of a dispersion and by the use of surfactants. Carrier formulations suitable for oral, subcutaneous, intravenous, intramuscular, etc. can be found in Remington's The Science and Practice of Pharmacy (2000).

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dose could be dissolved in 1 ml of isotonic NaCl solution and either added to 1000 ml of hypodermic or intravenous fluid or injected at the proposed site of infusion, (see, for example, Remington's Pharmaceutical Sciences, 15th Edition, pages 1035-1038 and 1570-1580).

In other embodiments, one may desire a topical application of compositions disclosed herein. Such compositions may be formulated in creams, lotions, solutions, gels, pastes, powders, or in solid form depending upon the particular application. The formulation of pharmaceutically acceptable carriers for topical administration is well known to one of skill in the art.

In another embodiment of the present invention, the therapeutic agent is formulated as a transdermal delivery device (“patches”). Such transdermal patches may be used to provide continuous or discontinuous infusion of the compounds of the present invention in controlled amounts. The construction and use of transdermal patches for the delivery of pharmaceutical agents is well known in the art. See, for example, U.S. Pat. No. 5,023,252, issued Jun. 11, 1991. Such patches may be constructed for continuous, pulsatile, or on demand delivery of pharmaceutical agents.

Other delivery systems can include time-release, delayed release or sustained release delivery systems. Such systems can avoid repeated administrations of the therapeutic agents of the present invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. No. 4,748,034 and U.S. Pat. No. 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Use of a long-term sustained release implant may be suitable for treatment of cholesterol-related disorders in patients who need continuous administration of the compositions of the present invention. “Long-term” release, as used herein, means that the implant is constructed and arranged to deliver therapeutic levels of the active ingredients for at least 30 days, and preferably 60 days. Long-term sustained release implants are well known to those of ordinary skill in the art and include some of the release systems described above.

In another embodiment of the present invention, the compound for treating high cholesterol comes in the form of a kit or package containing one or more of the therapeutic compounds of the present invention. These therapeutic compounds of the present invention can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the therapeutic compounds of the present invention. This drug delivery system can be used to facilitate administering any of the various embodiments of the therapeutic compounds of the present invention. In one embodiment, the system contains a plurality of dosages to be to be administered daily or weekly. The kit or package can also contain the agents utilized in combination therapy to facilitate proper administration of the dosage forms. The kits or packages also contain a set of instructions for the subject.

Without further elaboration, it is believed that one skilled in the art, based on the description herein, can utilize the present invention to its fullest extent. All publications recited herein are hereby incorporated by reference in their entirety. The following specific examples, which describe synthesis and biological testing of several compounds of this invention, are therefore to be construed as merely illustrative, and not limitative of the remainder of the disclosure in any way whatsoever.

### EXAMPLE 1

Synthesis

(1) Cholamide:

Cholamide, N-methyl-N-carboxymethyl-5β-cholanoic acid-24-amide, was prepared according to the reaction scheme below:

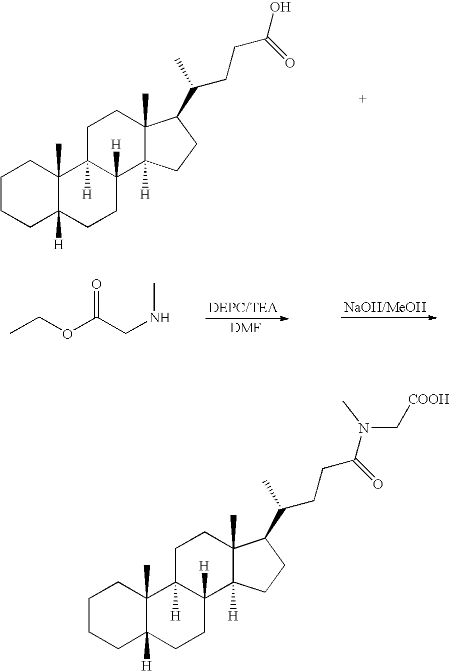

Specifically, 5β-cholanoic acid (100 mg) and sarcosine ethyl ester (100 mg) were dissolved in 1.5 ml dimethylformamide (DMF), to which 50 mg of diethyl pyrocarboniate (DEPC) was added, followed by 0.2 ml of triethylamine (TEA). The solution was stirred at 70° C. for 16 hours. The reaction mixture was diluted with water and extracted with ethylacetate. After washed sequentially with 1N HCl and 1N NaOH solution, ethylacetate was removed under reduced pressure to give a light yellow oil (80 mg). The oil was dissolved in 0.5 mL 0.5 N NaOH solution in methanol and stirred at 80° C. for 16 hours. The reaction mixture was diluted with water and extracted with ethylacetate. The ethylacetate layer was washed with 1N HCl and water. Ethylacetate was removed under reduced pressure to give cholamide (as white powder.

¹H-NMR (ppm): 0.648(3H), 0.913 (3H), 0.936-0.952 (3H), 3.11 (3H), and 4.09 (2H).

(2) Cholanosulfonic Acid

5β-cholanosulfonic acid, 3-(10,13-Dimethyl-hexadecahydro-cyclopenta[a]phenanthren-17-yl)-butane-1-sulfonic acid, was prepared by sulfonating a bile acid derivative having a haloalkyl substituent at C-17 of the ring system as depicted below:

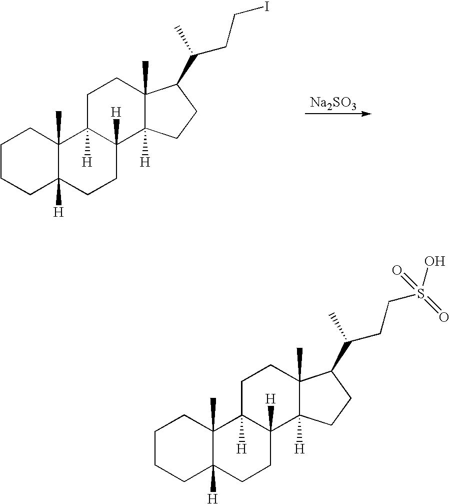

Specifically, 17-(3-iodo-1-methyl-propyl)-10,13-dimethyl-hexadecahydro-5β-cyclopenta[a]phenanthrene (100 mg) was dissolved in 30 ml ethanol, to which Na₂SO₃ (1.25 g) dissolved in 25 ml water was added. The mixture was refluxed for 10 hours. At the end, ethanol was distilled out. The water solution was left at 20° C. for 16 hours. The water solution was centrifuged and pellet was saved. The pellet was washed with water and dried to give cholanosulfonic acid.

(3) Δ^{3,5}-cholanoic acid

Δ^{3,5}-5β-cholanoic acid, 4-(10,13-Dimethyl-2,7,8,9,10,11,12,13,14,15,16,17-dodecahydro-1H-cyclopenta[a]phenanthren-17-yl)-pentanoic acid, was via a dehydroxylation reaction described in Chang et al., J. Am. Chem. Soc., 1957, 75, 4404. Specifically, 3α, 6α-dihydroxy-cholanoic acid 24-methyl ester (2 g) and p-toluensulfonic chloride were dissolved in 25 mL pyridine and stirred at 20° C. for 16 hours. After work-up, the product was re-dissolved in 30 mL 2,6-dimethylpyridine and heated under reflux for 6 hours. After cooling to 20° C., the reaction mixture was diluted with ice-water mixture and extracted with ethyl ether. The ethyl ether layer was collected and washed with 10% HCl three times, 8% NaHCO₃ one time, and water one time. Ethyl ether was removed under reduced pressure and the residue was re-dissolved in a KOH solution in methanol and stirred at 90° C. for 3 hours. After work-up, the final product Δ^{3,5}-cholanoic acid was collected.

(4) Additional Compounds

N-methyl-N-methoxy-5β-cholanoic acid-24-amide, N-carboxymethyl-5β-cholanoic acid-24-amide, N-sulfonylethyl-5β-cholanoic acid-24-amide, N-methyl-N-methylamino-5β-cholanoic acid-24-amide, N-formylamino-5β-cholanoic acid-24-amide, N-(2,2,2-trifluoroethyl)-5β-cholanoic acid-24-amide, N,N-dimethyl-5β-cholanioic acid-24-amide, N-methyl-5β-cholanoic acid-24-amide, N-ethoxycarbonylmethyl-5β-cholanoic acid-24-amide, and N-(2-chloroetlhyl)-5β-cholanoic acid-24-amide were prepared according to the method described above, except that hydroxy-containing compounds or other amine compounds, instead of sarcosine ethyl ester, were used.

### EXAMPLE 2

Reporter Gene Transactivation Assay

Human embryonic kidney 293 cells were seeded into 48-well culture plates at 2×104 cells per well in a Dulbecco's modified Eagle's medium (DMEM) supplemented with 10% fetal bovine serum. After 24 hours, cells were transfected by a calcium phosphate coprecipitation method with 250 ng of the pGL3/DR-4-luc reporter gene which consists of three copies of AGGTCAagccAGGTCA (DR-4) or pGL3/IR-1-luc which consists of three copies of GGGTCAcTGACCC (IR-1) fused to nucleotides −56 to +109 of the human c-fos promoter in front of the firefly luciferase gene in the plasmid basic pGL3 (Promega, Madison, Wis.), 40 ng pSG5/hRXRβ, 40 ng pR5/rFXR,10 ng pSG5/hGrip1, 0.4 ng CMV/R-luc (transfection normalization reporter, Promega) and 250 ng carrier DNA per well. This reporter does not have response elements for COUP-TFII or HNF4. After another 12-24 hours, cells were washed with PBS and refed with DMEM supplemented with 4% delipidated fetal bovine serum. Chemicals dissolved in ethanol were added in duplicate to the medium so that the final concentration of alcohol was 0.2%. After 24-48 hours, cells were harvested and luciferase activity was measured with a commercial kit (Promega Dual luciferase II) on a Monolight luminometer (Becton Dickenson, Mountain View, Calif.). FXR dimerizes with RXR to form an FXR/RXR heterodimer, which binds to both DR-4 response elements and IR-1 response elements in a ligand-dependent manner and transactivates the reporter gene expression.

The tested compounds include cholamide, cholanosulfonic acid, Δ^{3,5}-cholanoic acid, N-metlhyl-N-methoxy-5β-cholanoic acid-24-amide, N-carboxynethyl-5β-cholanoic acid-24-amide, N-sulfonylethyl-5β-cholanoic acid-24-amide, N-methyl-N-methylamino-5β-cholanoic acid-24-amide, N-formylamino-5β-cholanoic acid-24-amide, N-(2,2,2-trifluoroethyl)-5β-cholanoic acid-24-amide, N,N-dimethyl-5β-cholanoic acid-24-amide, N-methyl-5β-cholanoic acid-24-amide, N-ethoxycarbonylmethyl-5β-cholanoic acid-24-amide, and N-(2-chloroethyl)-5β-cholanoic acid-24-amide. The results show that all of these compounds were potent agonists of FXF.

For instance, cholamide exhibited unexpected higher efficacy (200 times) than GW4064, a known non-steroidal FXR agonist.

### EXAMPLE 3

Coactivator-Receptor Ligand Assay

A GST-rFXR fusion protein was expressed in E. coli strain BL21 using the expression plasmid pGEX. Cells were lysed by one cycle of freeze-thaw and sonication. The supernatant, obtained after centrifugation at 45,000 g for 1 hour, was incubated with glutathione-agarose at 4° C. for 10 minutes. The agarose was washed with a pH 7.5 binding buffer which contained 20 mM Hepes, 10 mM EDTA, 10 mM Na₂MoO₄, 1 mM b-mercaptoethaniol, 1 mM DTT, 0.5 mM PMSF, and 2 ug aprotinin/ml. Human Grip1 was produced by in vitro translation using a rabbit reticulocyte lysate and labeled with [³⁵S]-methionine. [³⁵S]-Grip1 in reticulate lysate (2 mL) was added to GST-UR bound to agarose beads in 100 uL binding buffer. An ethanol solution containing a compound to be tested was added to the mixture and the slurry was shaken at room temperature for 30 minutes. The agarose beads were then washed three times with binding buffer. Bound protein was eluted with SDS-PAGE loading buffer and separated on a 8% SDS-PAGE gel. Gels were dried and subjected to autoradiography. Radioactive Grip1 was measured with a STORM phosphoimager (Molecular Dynamics).

### EXAMPLE 4

Northern Blot Analysis

Male Fischer 344-HSD rats of 8-weeks old were used in the study. A low-fat chow diet was used throughout the study. A compound tobe tested was mixed with food at different concentrations for oral uptake. The average food consumption was 25 g/day/rat. Lighting conditions were controlled according to an alternating 12-hour light and 12-hour dark cycles (7 a.m.-7 p.m). At all the doses tested, there was no difference of food consumption due to various amounts of a compound of this invention. Animals were fasted for 12 hours before sacrifice. All of the tested compounds mentioned above were able to lower serum cholesterol and triglycerides.

Using liver tissue from the sacrificed rats, Northern Block analysis revealed that these tested compounds induced FXR gene expression in the liver. OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A compound of formula (I): 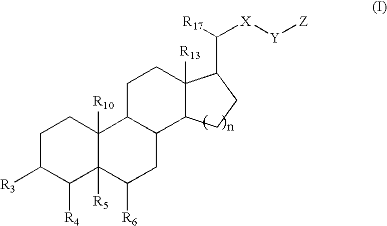
wherein
n is 1 or 2;
each of R₃, R₄, R₅, and R₆, independently, is hydrogen;
each of R₁₀, R₁₃, and R₁₇, independently, is hydrogen or C₁₋₄ alkyl;
X is C₁₋₈ alkylene, C₂₋₈ alkenylene, or C₂₋₈ alkynylene;
Y is —CO—, —CS—, or —CNH—; and
Z is C₁₋₈ alkyl or NRₐR_{b}, wherein each of Rₐ and R_{b}, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl.

2. The compound of claim 1, wherein n is 1; each of R₃, R₄, R₅, and R₆, independently, is hydrogen; and each of R₁₀, R₁₃, and R₁₇, independently, is methyl.

3. The compound of claim 2, wherein Y is —CO—.

4. The compound of claim 2, wherein X is C₁₋₈ alkylene.

5. The compound of claim 4, wherein Y is —CO—.

6. The compound of claim 4, wherein X is ethylene.

7. The compound of claim 6, wherein Y is —CO—.

8. The compound of claim 7, wherein Z is (methyl)(carboxymethyl)amino, (methyl)(methoxy)amino, (carboxymethyl)amino, (methylamino)(methyl)amino, (methylamino)(methyl)amino, (formylamino)amino, (2,2,2-trifluoroethyl)amino, dimethylamino, ethyoxycarbomethyl, or (2-chloroethyl)amino.

9. The compound of claim 8, wherein Z is (methyl)(carboxymethyl)amino.

10. The compound of claim 1, wherein X is C₁₋₈ alkylene.

11. The compound of claim 10, wherein Y is —CO—.

12. The compound of claim 11, wherein Y is —CO—.

13. A compound of formula (I): 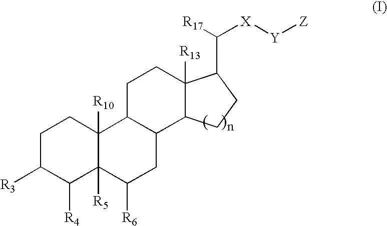
wherein
n is 1 or 2;
each of R₃, R₄, R₅, and R₆, independently, is hydrogen; or R₃ and R₄ together, or R₅ and R₆ together, are eliminated so that the carbon atoms to which they are attached are connected via a double bond;
each of R₁₀, R₁₃, and R₁₇, independently, is hydrogen or C₁₋₄ alkyl;
X is C₁₋₈ alkylene, C₂₋₈ alkenylene, or C₂₋₈ alkynylene;
Y is —SO—, —SO₂—, —PO—, or —PO₂—; and
Z is hydroxyl, C₁₋₈ alkyl, C₁₋₈ alkoxy, or NRₐR_{b}, wherein each of Rₐ and R_{b}, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl.

14. The compound of claim 13, wherein n is 1; each of R₃, R₄, R₅, and R₆, independently, is hydrogen; and each of R₁₀, R₁₃, and R₁₇, independently, is methyl.

15. The compound of claim 14, wherein Y is —SO₂—.

16. The compound of claim 14, wherein X is C₁₋₈ alkylene.

17. The compound of claim 16, wherein Y is —SO₂—.

18. The compound of claim 16, wherein X is ethylene.

19. The compound of claim 18, wherein Y is —SO₂—.

20. The compound of claim 19, wherein Z is hydroxy.

21. The compound of claim 13, wherein X is C₁₋₈ alkylene.

22. The compound of claim 13, wherein Y is —SO₂—.

23. A compound of formula (I): 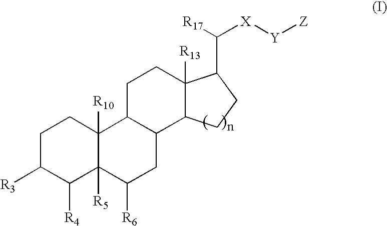
wherein
n is 1 or 2;
R₃ and R₄ together, or R₅ and R₆ together, are eliminated so that the carbon atoms to which they are attached are connected via a double bond;
each of R₁₀, R₁₃, and R₁₇, independently, is hydrogen or C₁₋₄ alkyl;
X is C₁₋₈ alkylene, C₂₋₈ alkenylene, or C₂₋₈ alkynylene;
Y is —CO—, —CS—, —CNH—, —SO—, —SO₂—, —PO—, or —PO₂—; and
Z is hydroxyl, C₁₋₈ alkyl, C₁₋₈ alkoxy, or NRₐR_{b}, wherein each of Rₐ and R_{b}, independently, is hydrogen, C₁₋₄ alkyl, C₁₋₄ alkoxy, C1-4 sulfonylalkyl, C₁₋₄ alkylamino, C₁₋₄ carboalkyl, or C₁₋₄ alkoxycarbonylalkyl.

24. The compound of claim 23, wherein n is 1; and each of R₁₀, R₁₃, and R₁₇, independently, is methyl.

25. The compound of claim 24, wherein X is ethylene; Y is —CO—; and Z is hydroxy.

26. A pharmaceutical composition comprising: an effective amount of a compound of formula (I): 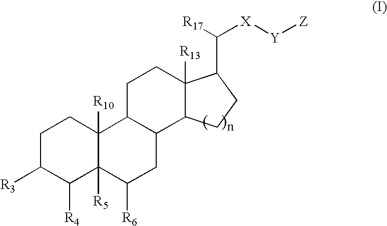
wherein
n is 1 or 2;
each of R₃, R₄, R₅, and R₆, independently, is hydrogen; or R₃ and R₄ together, or R₅ and R₆ together, are eliminated so that the carbon atoms to which they are attached are connected via a double bond;
each of R₁₀, R₁₃, and R₁₇, independently, is hydrogen or C₁₋₄ alkyl;
X is C₁₋₈ alkylene, C₂₋₈ alkenylene, or C₂₋₈ alkynylene;
Y is —CO—, —CS—, —CNH—, —SO—, —SO₂—, —PO—, or —PO₂—; and
Z is hydroxyl, C₁₋₈ alkyl, C₁₋₈ alkoxy, or NR₁R₂, wherein each of R₁ and R₂, independently, is hydrogen, C₁₋₄ alkyl, C₁₋₄ alkoxy, C₁₋₄ sulfonylalkyl, C₁₋₄ alkylamino, C₁₋₄ carboalkyl, or C₁₋₄ alkoxycarbonylalkyl; and
a pharmaceutically acceptable carrier.

27. The pharmaceutical composition of claim 26, wherein n is 1; each of R₃, R₄, R₅, and R₆, independently, is hydrogen; and each of R₁₀, R₁₃, and R₁₇, independently, is methyl.

28. The pharmaceutical composition of claim 27, wherein Y is —CO— or —SO₂—.

29. The pharmaceutical composition of claim 27, wherein X is C₁₋₈ alkylene.

30. The pharmaceutical composition of claim 29, wherein Y is —CO— or —SO₂—.

31. The pharmaceutical composition of claim 29, wherein X is ethylene.

32. The pharmaceutical composition of claim 31, wherein Y is —CO—.

33. The pharmaceutical composition of claim 32, wherein Z is (methyl)(carboxymethyl)amino, (methyl)(methoxy)amino, (carboxymethyl)amino, (methylamino)(methyl)amino, (methylamino)(methyl)amino, (formylamino)amino, (2,2,2-trifluoroetlhyl)amino, dimethylamino, ethyoxycarbomethyl, or (2-chloroethyl)amino.

34. The pharmaceutical composition of claim 33, wherein Z is (methyl)(carboxymethyl)amino.

35. The pharmaceutical composition of claim 31, wherein Y is —SO₂—.

36. The pharmaceutical composition of claim 35, wherein Z is hydroxy.

37. The pharmaceutical composition of claim 26, wherein X is C₁₋₈ alkylene.

38. The pharmaceutical composition of claim 37, wherein Y is —CO— or —SO₂—.

39. The pharmaceutical composition of claim 37, wherein X is ethylene.

40. The pharmaceutical composition of claim 38, wherein Y is —CO— or —SO₂—.

41. The pharmaceutical composition of claim 26, wherein Y is —CO— or —SO₂—.

42. The phainaceutical composition of claim 26, wherein R₃ and R₄ together, and R₅ and R₆ together, are eliminated so that the carbon atoms to which they are attached are connected via a double bond.

43. The pharmaceutical composition of claim 42, wherein n is 1; each of R₃, R₄, R₅, and R₆, independently, is hydrogen; and each of R₁₀, R₁₃, and R₁₇, independently, is methyl.

44. The pharmaceutical composition of claim 43, wherein X is ethylene; Y is —CO—; and Z is hydroxy.

45. A method of treating an FXR-mediated disease in a subject in need of such treatment, the method comprising administering an effective amount of a compound of claim 1 or 13 to the subject.

46. A method of treating an FXR-mediated disease in a subject in need of such treatment, the method comprising administering a pharmaceutical composition of claim 26 to the subject.

47. The method of claim 45, wherein the subject is a human.

48. The method of claim 46, wherein the subject is a human.
